(19) United States
(12) Patent Application Publication
(10) Pub. No.: US 2022/0280458 A1
(43) Pub. Date: Sep. 8, 2022

(54) Topical Composition for and Method of Preventing and Treating Pressure Sores and Skin Irritation

(71) Applicants: Margaret Elizabeth Meyers, Crowley, TX (US); Robert Michael Meyers, SR., Crowley, TX (US)

(72) Inventors: Margaret Elizabeth Meyers, Crowley, TX (US); Robert Michael Meyers, SR., Crowley, TX (US)

(21) Appl. No.: 17/191,090

(22) Filed: Mar. 3, 2021

(51) Int. Cl. A61K 31/19; A61K 9/00; A61K 9/12; A61K 36/886; A61K 47/46; A61K 36/87

(52) U.S. Cl. CPC A61K 31/19; A61K 9/0014; A61K 9/122; A61K 36/886; A61K 47/46; A61K 36/87

(57) ABSTRACT

Compositions of matter and methods for applying the compositions to a skin surface for prevention of and treatment of bed sores as a foamed topical cleanser including about one part by volume of diluted acetic acid (5% (w/v) aqueous solution); about one part by volume of Aloe leaf juice; and about at least ¼ part by volume of body wash concentrate; with the proviso that the composition have a consistency such that, when foamed by a dispenser, running due to gravity is prevented for a period of time sufficient for the composition to dry or to accomplish cleansing and conditioning prior to towel drying.

### FIELD OF THE INVENTION

This invention relates to topical compounds, lotions, ointments, liniments and medicines for the prevention and treatment of pressure and bed sores.

### BACKGROUND OF INVENTION

When a person or patient is immobile, such as being confined to a bed or chair, for an extended period of time, they may develop “bedsores” on their skin. Other names for bed sores which appear in common language and related literature are “pressure sores”, “pressure injuries”, “pressure ulcers”, and “decubitus ulcers”. Despite the name “bed” sore, these skin ulcers may occur to persons who use wheelchairs and even under casts for treatment of broken bones. Other medical conditions can make bed sores more likely or worse, such as age, nutrition, diabetes, circulation problems.

After a sore ruptures, repeated contact with the same area of bed sheets, clothing, and human waste can deepen the injury and prevent healing from beginning. Some bed sores extend beyond the skin and penetrate into muscle and even bone.

According to Johns Hopkins Hospitals, common treatments for bed sores include, but are not limited to, removing pressure from the injured area, protecting the injury with clean dressings which are changed often, maintaining cleanliness of the injury, improving nutrition, removing (debriding) the infected or dead tissue around the injury, transplanting healthy tissue to the injury, employing negative pressure to improve circulation to the injury, and use of various medicines such as antibiotics.

### EMBODIMENTS OF THE INVENTION

Disclosed are multiple embodiments of compositions of matter and methods for applying the compositions to a skin surface for prevention of and treatment of bed sores as a foamed topical cleanser including about one part by volume of diluted acetic acid (5% (w/v) aqueous solution); about one part by volume of aloe leaf juice; and about at least ¼ part by volume of body wash concentrate; with the proviso that the composition have a consistency such that, when foamed by a dispenser, running due to gravity is prevented for a period of time sufficient for the composition to dry or to accomplish cleansing and conditioning prior to towel drying.

### DETAILED DESCRIPTION OF ONE OR MORE EXEMPLARY EMBODIMENT(S) OF THE INVENTION

The present inventors have recognized that topical agents for preventing, treating and curing certain degrees of bed sores on patients, especially those patients who are being cared for in a non-hospital setting such as a hospice setting, are insufficient and ineffective.

While many techniques exist to try to prevent the development of bed sores, such as shifting the weight of the patient, repositioning them often, and changing their clothing and sheets often, bed sores still develop in many patients.

Despite the existence of dozens of over-the-counter and professional-grade topical solutions which purport to prevent, treat and heal bed sores in such settings, the present inventors, being caregivers of many at-home patients, have found none of them to be satisfactory for these purposes.

Therefore, for the purposes of the present disclosure, we will refer to bed sores, pressure sores, pressure injuries, pressure ulcers, and decubitus ulcers collectively as “bed sores”. The present inventors have found, through experimentation, research and practice, the following invention to be useful in early stages of bed sores (e.g., pre-rupture) and effective in moderately advanced stages of bed sores. Nothing contained in this patent disclosure should be used to override, replace or otherwise prevent following of any instructions or prescription given by a qualified healthcare provider such as a licensed medical doctor, registered nurse, etc. However, the following invention may be useful and effective as described, especially when no such instructions or prescription have been issued, but as with all medical and healthcare treatments, compounds, and practices, results may vary from patient to patient because no two patients are identical. For these reasons, the following example composition formulas and methods of using these compositions may be implemented within variations according to the best judgement of the care giver within the spirit and scope of the present invention.

Development of the present invention originated during care by the present inventors of persons who are in a sedentary position for prolonged periods of time in a non-hospital setting, such as home-based hospice care. As will be seen from the following paragraphs, there is nothing inherent to the present invention or its various embodiments which would prevent it from being useful in hospital settings, however, it is particularly effective in non-hospital, such as residential, settings.

The present inventors note that these patients often had one or more of the following health care challenges: incontinence of bowel or bladder, inability to reposition themselves, odor from incontinence, and rashes occurring under skin folds, arm pits and the breast. Some bedsores are detectable and noticeable in their very early stages simply as rashes or redness indicating a developing problem.

The present inventors, as highly experienced home health caregivers, were in need of a topical solution that would clean and condition the skin in these areas, which would promote healing, which is able to be applied without rinsing in a setting such as a bed or chair, and which would help eliminate or control odor.

The present inventors tried items that were made available to them from various pharmaceutical and healthcare product lines, but they did not fulfill these needs well. Some of the shortcomings included the way that the existing products were administered using regular spray bottles which could startle the patient, the consistency of the solutions being to thin and runny and therefore would dampen or soak bedding, the inability to remove urine and feces effectively and quickly, and the inability to eliminate unwanted odors along with sustaining and nourishing the skin.

Therefore, the present inventors embarked on an effort to develop one or more solutions which could meet all of these requirements. An early attempt in creating something that would meet these needs was a solution of water mixed with a retail liquid no-rinse soap per instructions for dilution on the product packaging. This was too thin and runny for in-bed or in-chair use, and did not appear to promote skin recovery, although it purports to contain aloe vera and to be antimicrobial.

Applicant's then added acetic acid, such as diluted apple cider vinegar, to the solution to increase antimicrobial properties, and tried applying this water-soap-vinegar composition to patients using a container with a foaming dispenser on it. However, the water-soap-vinegar solution, was still too thin and runny for use on a patient in a bed or chair without dampening or soaking bedding or clothing. Further, it was concerning to the present inventors that such thin solutions would spend too little time in contact with the treatment area to be effective. It was noticed, however, that using a container with a foam-generating dispenser appeared to solve the issue of startling the patient, which is one of the unmet needs to keep from waking a sleeping or sedated patient.

Subsequent experiments included using a reduced amount of water in the solution, adding 100% Aloe leaf extract juice to increase the healthy therapeutic effect and to further thicken the solution, and use of thicker no-rinse soap, such as t Aloe Vesta Body Wash and Shampoo manufactured by ConvaTec®, in the container with the foam-generating dispenser. This composition formulation resulted in a thicker foam, which could be described as a mousse, which allowed the applied solution to stay in place on the treatment area for a longer time.

In fact, the thickness of the foam in this experimental embodiment was sufficient that it could be allowed to air dry in place, which maximized application time of the ingredients to the treatment area and therefore the health benefits. Further, it was noticed that the increased time of contact of the foaming solution increased the amount of lift of residual feces and urine when used in areas subject to irritation due to incontinence, ostomy appliances, etc. The latter beneficial action also helped to clean the skin while breaking down human waste so that removal was easier.

Still further revisions and trials of composition variations included solutions in which the apple cider vinegar was replaced with 5% distilled white vinegar, which was observed through trial use to be correlated with quicker healing of an injured area. This particular solution also presented additional positive benefits including a noticeable preventative and healing results for areas where skin rashes appeared, such as in skin folds, sensitive areas, breaks in the skin, fungal infections, etc.

Due to the biting scent of vinegar left in place after application, the present inventors experimented with multiple essential oil additions to mask the acidic aroma. Lavender proved the most effective in reducing the scent of vinegar after application. Other essential oils that were tried which also reduced the unpleasant vinegar scent were citrus, thieves, rosemary, tea tree, peppermint, and chamomile.

Still other embodiment options according to the present invention include addition of vitamins, such as vitamin A and vitamin D, to bring further positive benefits in terms of promoting skin health and resiliency.

As such, according to the inventors' trials and combinations, a formula consisting of Aloe Barbadnesis Leaf Juice, distilled 5% acetic acid, water, disodium cocoamphodiacetate, sorbital, sodium C14-16, olefin sulfonate, PPG-2 hydroxyethyl cocamide, acrylates/C10-30 alkyl acrylate crosspolymer, styrene/acrylates copolymer, fragrance, methylparaben, propylparaben, FD&C Green No. 3, Vitamin A & D worked well for the aforementioned purposes and needs. Please note that Aloe Barbadnesis may go be referred to by other names or may be substituted with other variants of the aloe plant known as aloe vera, Aloe africana, Aloe arborescens, Aloe barbadensis, Aloe Capensis, Aloe ferox, Aloe frutescens, Aloe Gel, Aloe indica, Aloe Latex, Aloe Leaf Gel, Aloe natalenis, Aloe Perfoliata, Aloe perryi, Aloe spicata, Aloe supralaevis, Aloe ucriae, Aloe Vera Barbenoids, Aloe Vera Gel, Aloe vera, Aloes, Aloès, Aloès de

Curaçao, Aloès des Barbades, Aloès du Cap, Aloès Vrai, Aloès Vulgaire, Arborescens natalenis, Barbados Aloe, Burn Plant, Cape Aloe, Chritkumari, Curacao Aloe, Elephant's Gall, Gel de la Feuille d'Aloès, Ghee-Kunwar, Ghi-Kuvar, Ghrita-Kumari, Gvar Patha, Hsiang-Dan, Indian Aloe, Jafarabad Aloe, Kanya, Kidachi Aloe, Kumari, Latex d'Aloès, Lily of the Desert, Lu-Hui, Miracle Plant, Plant of Immortality, Plante de l'Immortalité, Plante de la Peau, Plante de Premiers Secours, Plante Miracle, Plantes des Brûlures, Sábila. We will refer to all of these name and variations collectively as “aloe” for the purposes of this disclosure.

In particular, according to at least a first embodiment of the present invention, the following three ingredients are combined in a 1:1:1 ratio which can be scaled to produce more or less of the useful composition for use via a foam-generating dispenser, in no particular order, as follows:
1 part of aloe leaf juice, such as but not limited to Aloe Barbadnesis;
1 part of acetic acid diluted to 5% with water, such as but not limited to distilled white vinegar;
1 part of concentrated, thick, mild convalescent body wash such as, but not limited to, Aloe Vesta Body Wash and Shampoo available from ConvaTec® (nominally containing Water (aqua), disodium cocoamphodiacetate, sorbitol, sodium C14-16 olefin sulfonate, PPG-2 hydroxyethyl cocoamide, acrylates/C10-3-akyl acrylate crosspolymer, styrene/acrylates copolymer, fragrance, methylparaben, aloe barbadensis leaf juice, polyparaben, FD&C green No. 3); and
optional vitamins, such as but not limited to vitamins A and D, and masking fragrances, such as but not limited to lavender, citrus, thieves, rosemary, tea tree, peppermint, and chamomile.

In this first example embodiment, when the measuring part is set to 2²/₃ ounces, the amount of solution yielded is approximately 8 ounces, which is convenient for many available containers having a foam-generating dispenser.

According to at least a second embodiment of the present invention, the following three ingredients are combined in a 7:7:2 ratio which can be scaled to produce more or less of the useful composition for use via a foam-generating dispenser, in no particular order, as follows:
7 parts of aloe leaf juice, such as but not limited to Aloe Barbadnesis;
7 parts of acetic acid diluted to 5% with water, such as but not limited to distilled white vinegar, or Spectrum™ acetic acid available from Fisher Scientific®, 5% (w/v) Aqueous Solution;
2 parts of concentrated, thick, mild convalescent body wash such as, but not limited to, Aloe Vesta Body Wash and Shampoo available from ConvaTec® (nominally containing Water (aqua), disodium cocoamphodiacetate, sorbitol, sodium C14-16 olefin sulfonate, PPG-2 hydroxyethyl cocoamide, acrylates/C10-3-akyl acrylate crosspolymer, styrene/acrylates copolymer, fragrance, methylparaben, aloe barbadensis leaf juice, polyparaben, FD&C green No. 3); and
optional vitamins, such as but not limited to vitamins A and D, and masking fragrances, such as but not limited to lavender, citrus, thieves, rosemary, tea tree, peppermint, chamomile.

In this second example embodiment, when the measuring part is set to one-half (¹/₂) ounce, the amount of solution yielded is approximately 8 ounces, which is convenient for many available containers having a foam-generating dispenser.

As such, the present inventors believe that solutions ranging from equal parts of aqueous 5% acetic acid and Aloe combined with as little as ¹/₄ part and as much as one part of concentrated, thick, mild convalescent body wash applied as a mousse-like foam are suitable embodiments of the present invention to meet the foregoing needs.

Regarding method of usage of the present invention, there are a range of methods which fall within the present invention and which can be employed according to the best judgement of the healthcare professional using the composition. For rashes and areas of skin showing irritation such as redness, but which are not yet open sores, preventative therapy includes the following steps:

(a) remove bulk waste matter from incontinence (feces, urine) as necessary;

(b) apply foamed solution prepared according to the foregoing paragraphs to the area of the rash or irritation, spreading the foam to cover the area with a gloved hand, wet wipe, or other suitable clean cloth or towel;

(c) allow foam to work for a few seconds to minutes to lift residual waste matter from the skin using the bubbling action;

(d) allow the foam to air dry, or pat dry with clean paper towel or cloth towel; and

(e) optionally, apply a thin layer of barrier cream, such as Lantiseptic® lanolin-based moisture skin protectant available from DemarRite Industries, LLC®.

For bed sores which are open or more advanced, restorative and protective therapy includes the following steps:

(a) remove bandage or dressing and bulk waste matter from incontinence (feces, urine) as necessary;

(b) apply foamed solution prepared according to the foregoing paragraphs to the area of the rash or irritation, spreading the foam to cover the area with a gloved hand, wet wipe, or other suitable clean cloth or towel;

(c) allow foam to work for a few seconds to minutes to lift residual waste matter from the skin using the bubbling action;

(d) allow the foam to air dry, or pat dry with clean paper towel or cloth towel;

(e) optionally, apply a thick layer of barrier cream, such as Lantiseptic® lanolin-based moisture skin protectant available from DemarRite Industries, LLC®; and

(f) optionally, apply fresh, clean bandage or dressing as necessary.

Typically, performing the foregoing methods of usage one or twice per day is sufficient to prevent, treat, heal and protect many bed sores in their various stages of presentation.

Conclusion. The terminology used herein is for the purpose of describing particular exemplary embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof, unless specifically stated otherwise.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Certain embodiments utilizing a microprocessor executing a logical process may also be realized through customized electronic circuitry performing the same logical process(es). The foregoing example embodiments do not define the extent or scope of the present invention, but instead are provided as illustrations of how to make and use at least one embodiment of the invention.

What is claimed is:

1. A method comprising:
applying, through a foaming dispenser, to a skin surface, a topical cleansing, antimicrobial and conditioning composition comprising:
(a) about one part by volume of acetic acid, 5% (w/v) aqueous solution;
(b) about one part by volume of Aloe leaf juice; and
(c) about at least ¼ part by volume of body wash concentrate;
with the proviso that the composition is of a consistency to prevent running due to gravity for a period of time sufficient to dry or to accomplish cleansing and conditioning prior to towel drying.

2. The method of claim 1 wherein the composition comprises about one part of the body wash concentrate.

3. The method of claim 1 wherein the acetic acid comprises diluted white vinegar.

4. The method of claim 1 wherein the acetic acid comprises diluted apple cider vinegar.

5. The method of claim 1 wherein the body wash concentrate comprises Aloe Vesta Body Wash and Shampoo available from ConvaTec®.

6. The method of claim 1 wherein the body wash concentrate comprises water (aqua), disodium cocoamphodiacetate, sorbitol, sodium C14-16 olefin sulfonate, PPG-2 hydroxyethyl cocoamide, acrylates/C10-3-akyl acrylate crosspolymer, styrene/acrylates copolymer, methylparaben, and polyparaben.

7. The method of claim 1 further comprising (d) an amount of essential oil sufficient to mask an odor from the acetic acid.

8. The method as set forth in claim 7 wherein the essential oil comprises lavender oil.

9. The method as set forth in claim 7 wherein the essential oil comprises citrus.

10. The method as set forth in claim 7 wherein the essential oil comprises thieves oil.

11. The method as set forth in claim 7 wherein the essential oil comprises rosemary oil.

12. The method as set forth in claim 7 wherein the essential oil comprises tea tree oil.

13. The method as set forth in claim 7 wherein the essential oil comprises peppermint oil.

14. The method as set forth in claim 7 wherein the essential oil comprises chamomile oil.

15. A composition of matter for applying to a skin surface as a foamed topical cleanser comprising:
(a) about one part by volume of acetic acid, 5% (w/v) aqueous solution;
(b) about one part by volume of Aloe leaf juice; and
(c) about at least ¼ part by volume of body wash concentrate;
with the proviso that the composition have a consistency such that, when foamed by a dispenser, running of the composition due to gravity is prevented for a period of time sufficient for the composition to dry or to accomplish cleansing and conditioning prior to towel drying.

16. The composition of claim 15 wherein the composition the body wash concentrate comprises about one party by volume.

17. The composition of claim 15 wherein the acetic acid comprises diluted white vinegar, or diluted apple cider vinegar, or both diluted white vinegar and diluted apple cider vinegar.

18. The composition of claim 15 wherein the body wash concentrate comprises Aloe Vesta Body Wash and Shampoo available from ConvaTec®.

19. The composition of claim 15 wherein the body wash concentrate comprises water (aqua), disodium cocoamphodiacetate, sorbitol, sodium C14-16 olefin sulfonate, PPG-2 hydroxyethyl cocoamide, acrylates/C10-3-akyl acrylate crosspolymer, styrene/acrylates copolymer, methylparaben, and polyparaben.

20. The composition of claim 15 further comprising (d) an amount of essential oil sufficient to mask an odor from the acetic acid comprising one or more essential oils selected from the group consisting of lavender oil, citrus, thieves oil, rosemary oil, tea tree oil, peppermint oil, and chamomile oil.
